(19) United States
(12) Patent Application Publication
(10) Pub. No.: US 2021/0220039 A1
(43) Pub. Date: Jul. 22, 2021

(54) UTERUS MANIPULATION DEVICE

(71) Applicant: ONDOKUZ MAYIS UNIVERSITESI REK-TORLUK, Samsun (TR)

(72) Inventor: Abdulkadir BAKAY, Samsun (TR)

(73) Assignee: Ondokuz Mayis Universitesi, Samsun (TR)

(21) Appl. No.: 17/051,430

(22) PCT Filed: May 10, 2019
(86) PCT No.: PCT/TR2019/050320

§ 371 Date: Oct. 28, 2020

(30) Foreign Application Priority Data
May 14, 2018 (TR) 2018/06807

(51) Int. Cl. A61B 18/14; A61B 18/00

(52) U.S. Cl. CPC A61B 18/14; A61B 2018/00601; A61B 2018/00285; A61B 2018/00559; A61B 2018/1425

(57) ABSTRACT

The present invention relates to uterus manipulator placed in the beginning of the sure for the purpose of facilitating cutting of uterus and its related bonds without causing any damage to other tissues in the hysterectomy procedure that is implemented laparoscopically and laparoscopically stitching of the opening occurred afterwards. The invention, particularly, relates to uterus manipulation device facilitating manipulation of the uterus, cutting every sort of tissue connected to the uterus, cutting cervicovaginal tissue in a circular line by way of using electrical energy by the device and stitching intracorporeally the opening, that will occur, by means of the device.

### RELATED TECHNICAL FIELD

The present invention relates to uterus manipulator placed in the beginning of the surgery for the purpose of facilitating cutting of uterus and its related bonds without causing any damage to other tissues in the hysterectomy procedure that is implemented laparoscopically (devices and monitoring system placed through holes with diameters of 0.5 mm or lcm into stomach) and laparoscopically stitching of the opening occurred afterwards.

The invention, particularly, relates to uterus manipulation device facilitating manipulation of the uterus, cutting every sort of tissue connected to the uterus, cutting cervicovaginal tissue in a circular line by way of using electrical energy by the device and stitching intracorporeally the opening, that will occur, by means of the device.

### STATE OF THE ART

During the procedure of hysterectomy, surgeon separates uterus section from ovaries, fallopian tubes, upper portion of the vagina, blood vessels and connective tissue and the procedure may be performed abdominally or vaginally.

This procedure has been started to be implemented laparoscopically with the development of the technology. Laparoscopic hysterectomy has become the more preferred procedure. Uterus manipulators are employed in the operations of laparoscopic hysterectomy.

Uterus manipulators are used to help that main steps of the surgery are performed more easily and to reduce possible damage to other organs by way of ensuring that the uterus is moved in a desired direction of the surgeon in the course of the procedure.

Since the processes of cutting cervicovaginal junction circularly and stitching the opening occurred, being the last steps of the surgery, is related directly to the experience and skill of the surgeon in an increasing rate according to the procedure types (type 1, 2 and three), the procedure may last longer in these steps, the possibility pertaining to damaging other organs increases and it may cause particularly less experienced surgeons not to prefer the laparoscopic hysterectomy procedure, of which advantages are proven scientifically.

The patent document numbered “US20100280524” has been examined within the result of the preliminary survey conducted in the state of the art. Said application discloses an apparatus used in hysterectomy surgeries. Since stitching process is carried out with said apparatus disclosed by the application after cutting process, this both causes the surgery to last longer and causes additional processes in terms of surgeon.

The patent document numbered “U.S. Pat. No. 5,275,613” has been examined within the result of the preliminary survey conducted in the state of the art. Said application discloses an apparatus used in hysterectomy surgeries and employed to cut cervix. The cutting and stitching processes are performed by separate devices with said invention.

The patent document numbered “US2010106163” has been examined within the result of the preliminary survey conducted in the state of the art. Said application discloses an apparatus fixed by inserting inside uterus. In said invention, it can be seen a cervical fixing portion and balloon and elongate shaft system inserting in the uterus.

After preliminary research about the state of the art, the patent document numbered “U.S. Pat. No. 9,717,524B2” has been examined. Said application discloses a device in an arm form so as to close vaginal cuff of the vaginal canal subsequent to operations like hysterectomy, in which the cervix is removed.

The patent document numbered “US2009137970” has been examined within the result of the preliminary survey conducted in the state of the art. Said application discloses variable universal laparoscopic uterus manipulators/dilatators, such that they comply with the length of the uterus, so as to avoid perforation risk and to provide a perfect pelvic exposure regardless of uterus size, shape and position.

The patent document numbered “US2015080905” has been examined within the result of the preliminary survey conducted in the state of the art. Said application discloses a cutting device used in surgical procedures such as laparoscopic hysterectomy, which ensures to cut from the vaginal side inwards, instead of cutting from the abdominal side outwards of the uterus during the process for taking a part from the uterus.

Uterus manipulators used in the state of art bring along various problems particularly in terms of cutting cervicovaginal junction and stitching the opening occurred.

When examining apparatuses employed in the state of art, there is no development distinguishing itself in a more attractive way from other products particularly in the subject of cutting cervicovaginal junction and stitching the opening occurred.

As a result, due to the aforementioned drawbacks and insufficiency of the existing solutions regarding the subject matter, the need for a development in the related technical field has arisen.

### OBJECT OF THE INVENTION

Main object of the invention is to determine the opening that will occur subsequent to cutting cervicovaginal junction in the laparoscopic hysterectomy surgery before cutting process and to fix it circularly by applying at the desired amount of stitching.

Another object of the invention is to ensure that the cervicovaginal tissue is cut in a circular line with the same needle by using electrical energy subsequent to the fixing process and the opening resulting from the extraction of the sample is closed in a single step. Thus, the duration of surgery is shortened, and the stitching process is implemented prior to the cutting process, thereby avoiding possible complications and difficulties that may be faced in the cutting and stitching process.

Another object of the invention is to ensure to perform the surgery easily by providing the movement of uterus in two planes (transversal and vertical).

Another object of the invention is to ensure that both cutting and stitching processes are performed by means of a single device.

A further object of the invention is to make it possible that a cervix fixing system is used with, of which width is adjustable by means of a fan system.

The structural and characteristic aspects and all the advantages of the present invention will be more clearly understood by means of following figures and the detailed description written with references to these figures. Therefore, assessment should be done by taking these figures and detailed description into consideration.

### DESCRIPTION OF THE FIGURES

FIG. 1 illustrates a side perspective view of the inventive manipulator mechanism in a mounted manner.

FIG. 2 illustrates a view of the main body and movable sled system of the inventive uterine manipulator.

FIG. 3 illustrates a view of the double-articulated adaptive head of the inventive uterine manipulator.

FIG. 4 illustrates a top view of the adaptive system and needle chamber of the inventive uterine manipulator.

FIG. 5 illustrates a detailed view of the needle of the inventive uterine manipulator.

### REFERENCE NUMBERS

100. Uterus Manipulation Device
110. Upper Balloon
120. Main Body Movable Outer Tube
   121. Lower Balloon
130. Main Body Movable Interior Tube
140. Adaptive Movable Heads
150. Adaptive Movable Head Needle Chamber
160. Articulations
170. Needle
   171. Needle Pointed End
   172. Needle Cutter Portion
   173. Needle Base

### DESCRIPTION OF THE INVENTION

The inventive uterus manipulation device (100), in general, is comprised of upper balloon (110), main body movable outer tube (120), lower balloon (121) connected thereto, main body movable interior tube (130), adaptive movable heads (140), adaptive movable head needle chamber (150), articulations (160) and needle (170). Needle (170) is comprised of needle pointed end (171), needle cutter portion (172) and needle base (173).

It is to ensure by means of the inventive uterus manipulation device (100) that the cervicovaginal tissue is cut in a circular line with the same needle by using electrical energy subsequent to the fixing process and the opening resulting from the extraction of the sample is closed in a single step. Thus, the duration of surgery is shortened, and the stitching process is implemented prior to the cutting process, thereby avoiding possible complications and difficulties that may be faced in the cutting and stitching process.

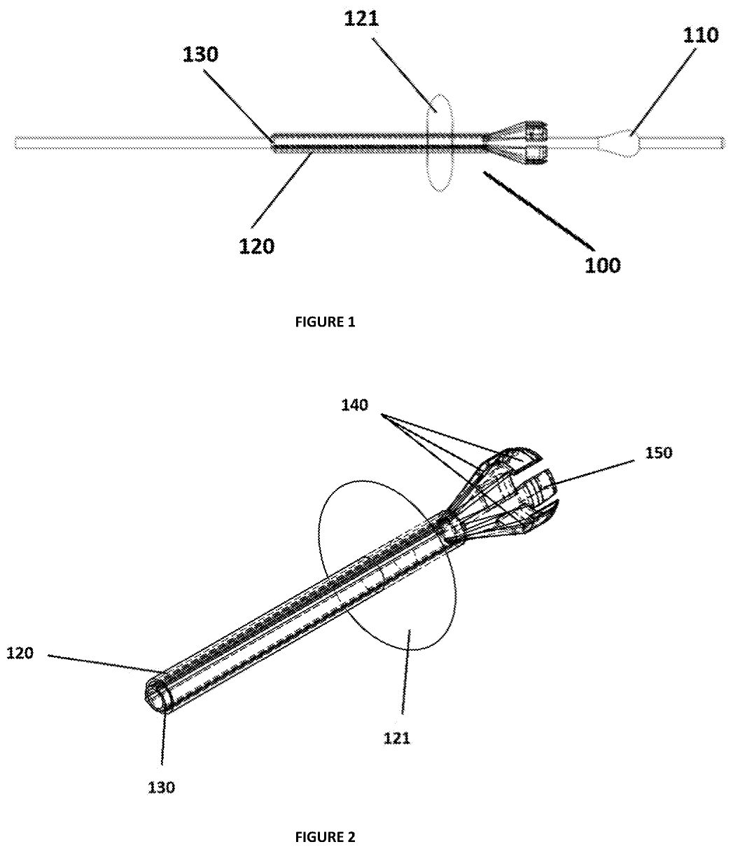

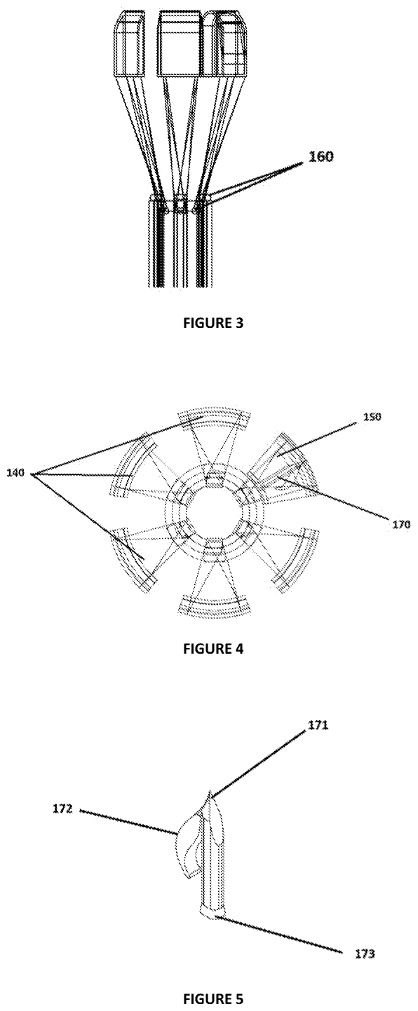

There is the shaft structure, which extends related to upper balloon (110) shown in FIG. 1 and may move horizontally in the main body movable interior tube (130), in the inner portion of the uterus manipulation device (100). Main body movable outer tube (120) and main body movable interior tube (130) constitute the main body of the uterus manipulation device (100). Main body movable outer tube (120) and main body movable interior tube (130) are connected to each other through a sled system and they can move over each other. There is a lower balloon (121) that is in a stable position on the main body movable outer tube (120) and prevents air and gas outlet during surgery.

There are adaptive movable heads (140) and adaptive movable head needle chamber (150), that surround the cervix from outside at the end portion shown in FIG. 2 of the uterus manipulation device (100) and have a fan form and of which diameter can be varied by means of articulations. (160). Said adaptive movable heads (140) may be opened and closed by means of articulations (160) by means of allowing the body movable outer tube (120) and main body movable interior tube (130) for moving on each other.

Uterus manipulation device (100) comprises at least one adaptive movable head (140) and at least one adaptive movable head needle chamber (150). There is a needle (170) in the adaptive movable head needle chamber (150). The needle (170) has a different structure than those adaptive movable heads (140).

The needle (170) is comprised of a needle pointed end (171) with a boring function, a need cutter portion (172) that is to perform the cutting process when loaded with electricity and fixing needle base (173). The needle (170) can be taken out of the adaptive movable head needle chamber (150) by means of elevating it upwards from the needle base (173), if desired. There is a spring under the needle (170) in a closed position. Said spring is opened by means of loosening the string on the control arm. Thus, it is ensured that the needle (170) is moved out of the adaptive movable head needle chamber (150). Thus, the needle (170) that can bore may perform the stitching process prior to the cutting process by means of taking out of the uterus manipulation device (100), if desired. The cutting process is performed by means of the need cutter portion (172) by way of providing electricity to the needle (170) through the need base (173). Said process is carried out by means of taking the needle (170) out of the adaptive movable head needle chamber (150) and rotating it around its own axis.

The operation performed by way of using the uterus manipulation device (100) is described as below.

Before the surgery, the material (string) to be used in stitching is placed to the needle (170) shown in FIG. 5 such that it is inserted through the needle cutter portion (172). Afterwards, the upper balloon (110) end of the uterus manipulation device (100) is placed in the uterus by inserting through the cervix widened in advance by using bougies. Upper balloon (110) is inflated with 5 cc of air and the uterus manipulation device (100) is fixed into the uterus. The adaptive movable head (140) is pushed forward to the cervix. Adaptive movable heads (140) and adaptive movable head needle chamber (150) are adjusted and locked to the cervix by means of articulations (160) according to size of cervix. The adaptive movable head (140) having the needle (170) therein is brought to 15° of the angular alignment of the cervicovaginal junction in planar with a rotational movement by means of the surgeon's method. By means of the movement transmission system of the spring mechanism, the needle (170) in the adaptive movable head needle chamber (150) carries the stitching material placed in the needle cutter portion (172) in advance into the intra-abdominal section by boring the cervicovaginal junction from 15° of the angular alignment in planar. Surgeon frees the stitching material from the position shown in the needle cutter portion (172) of the needle (170) by means of using a holder tool. The needle is drawn back (170) by using the same spring system and is brought to 345° of the angular alignment in planar with a rotational movement and is re-carried into the intra-abdominal section as empty. Surgeon places the stitching material in the needle cutter portion (172) of the needle (170). The needle (170) with the stitching material is drawn back with the same spring mechanism and brought to 105° of the angular alignment in planar with a rotational movement and thus, the stitching material is carried. After the stitching process is carried out with the same process in 105°, 75° and 45° of the angular alignment in planar, respectively, and the stitching process completed, and free ends of the stitching material is fixed by taking them out of the vagina. The lower balloon (121) made up of silicon and similar materials is inflated. After adaptive movable heads (140) are proceeded for almost 1 cm, the adaptive movable head needle chamber (150) is brought to 0° of the angular alignment in planar of the cervicovaginal junction. The needle (170) is re-carried into the intra-abdominal section by using the same spring system. The electrical energy, to which the needle (170) is connected, is transmitted by means of pressing on switch completing the circuit on the uterus manipulation device (100) and is rotated by transmitting the electrical energy with a rotational movement as a whole, until the cervicovaginal junction is cut completely. After this process is completed, the lower balloon (121) is deflated and discharged through the uterus-vaginal way along with the uterus manipulation device (100). The operation is ended, after free ends of the stitching material fixed outside in advance are tied.

It is to ensure by means of the inventive uterus manipulation device (100) that the cervicovaginal tissue is cut in a circular line with the same needle (170) by using electrical energy and the opening resulting from the extraction of the sample is closed in a single step. Further, the stitching process is carried out before the cutting process, thereby avoiding possible complications and difficulties that may be faced in the cutting and stitching process.

1. Uterus manipulation device (100) placed through the vagina in the beginning of the operation so as to cut the uterus and its related bonds in the hysterectomy procedure implemented laparoscopically and to stitch laparoscopically the opening occurred afterwards, characterized by comprising:
Main body movable outer tube (120) that is in a stable position on the lower balloon (121) preventing air and gas outlet during the surgery,
Adaptive movable heads (140), that surround the cervix from outside, have a fan structure and of which diameter can be varied by means of articulations (160), wherein the body movable outer tube (120) and main body movable interior tube (130) move over each other,
Adaptive movable head needle chamber (150), that has a needle (170) therein and allows said needle (170) for protruding outwards,
Needle (170), that accommodates the need cutter portion (172) performing the cutting process and the fixing need base (173) thereon, when the needle pointed end (171) with a boring function is loaded with electricity and allows the stitching process to be carried out prior to the cutting process.

2. (canceled)

3. Uterus manipulation device (100) according to claim 1, characterized by comprising a movement transmission system allowing the uterus to move on two planes during the procedure.

4. Uterus manipulation device (100) according to claim 1, characterized by comprising the needle (100) that can be taken out of the adaptive movable head needle chamber (150) by means of elevating it upwards from the need base (173).

5. Uterus manipulation device (100) according to claim 1, characterized by comprising a spring that is in closed position under the lower portion of the needle (170) and that can be brought to the open position by loosening the string on the control arm and that allows the needle (170) to move out of the adaptive movable head needle chamber (150).

6. Uterus manipulation device (100) according to claim 1, characterized by comprising a structure allowing main body movable outer tube (120) and main body, movable interior tube (130) that are connected to each other through a sled system to move over each other.
